(19) United States
(12) Patent Application Publication
(10) Pub. No.: US 2024/0399072 A1
(43) Pub. Date: Dec. 5, 2024

(54) PORTABLE LIGHT-GUIDED INJECTION DEVICE

(71) Applicant: SOLMEDIX CO., LTD., Seoul (KR)

(72) Inventors: Kwon Soo CHUN, Bucheon-si, Gyeonggi-do (KR); Ho Chul KANG, Goyang-si, Gyeonggi-do (KR); Inchul YANG, Sejong-si (KR)

(73) Assignee: SOLMEDIX CO., LTD., Seoul (KR)

(21) Appl. No.: 18/269,962

(22) PCT Filed: Dec. 29, 2021
(86) PCT No.: PCT/KR2021/020110

§ 371 Date: Jun. 19, 2024

(30) Foreign Application Priority Data
Dec. 29, 2020 (KR) 10-2020-0186760

(51) Int. Cl. A61M 5/32

(52) U.S. Cl. CPC A61M 5/3287; A61M 2205/587; A61M 2210/065

(57) ABSTRACT

A portable light-guided injection device according to an embodiment of the present invention may include a syringe part that injects drug; an injection needle part that invades an affected portion and injects the drug into the body; a main body part that connects the syringe part and the injection needle part to provide a drug transmitting path and has a light generating part configured to provide light to the affected portion; and an optical cable part that transmits the light provided from the light generating part to the affected portion.

### TECHNICAL FIELD

This application pertains to a portable device for light-guided injections.

### BACKGROUND

Vocal cord paralysis is a condition that occurs due to recurrent laryngeal nerve damage, which can be caused by various factors, including surgical procedures (such as those involving the thyroid, lung, esophagus, heart, head and neck, spine, and brain), cancer invasion (such as thyroid cancer, lung cancer, esophageal cancer, and head and neck cancer), and traumatic incidents (such as traffic accident).

Recently, there has been a rise in the frequency of surgeries and cancers that are recognized as potential causes of vocal cord paralysis. Therefore, many patients suffer from complications such as voice disorders, dysphagia, and aspiration pneumonia.

When vocal cord paralysis results in a less severe voice disorder, it can be effectively managed through voice therapy or rehabilitation. However, in many cases, surgical treatment is necessary to address the condition.

The primary surgical treatment options for vocal cord paralysis are centered on procedures performed under general anesthesia or invasive surgeries that involve external skin incisions, such as thyroid cartilage plastic surgery or arytenoid adduction.

Recently, there has been growing attention on a simple and effective non-invasive surgical method called vocal cord injection, thanks to the advancements in bio-injection materials and the improvements in material stability.

Vocal cord injection is primarily employed as a primary treatment for unilateral vocal cord paralysis and spasmodic dysphonia. However, it still has certain drawbacks concerning the ease and precision of the procedure.

The precise positioning of percutaneous vocal cord injection is currently reliant on the operator's skills and experience, as there is no technology or equipment available to assist in this regard.

Acquiring proficiency in percutaneous vocal cord injection usually requires extensive experience working with numerous patients. The learning curve associated with this procedure is relatively steep, resulting in a high entry barrier for individuals with limited experience.

Education and training programs for vocal cord injection are being conducted to assist non-experts in addressing the challenges associated with accurately positioning anatomically complex objects.

To overcome the limitations of percutaneous vocal cord injection, a light-guided injection device has been developed (see Patent Document 1).

The light-guided injection device, as disclosed in Patent Document 1, comprises light source equipment and consumables, including optical fiber and injection needle. During the percutaneous vocal cord injection, an LED light source is employed to illuminate the affected area through the optical fiber, facilitating the positioning of the injection needle and enabling drug infusion to the target site simultaneously.

However, the light-guided injection device disclosed in Patent Document 1 presents the following issues.

During the vocal cord injection procedure, an assistant is necessary to support the operator in securing a clear view of the vocal cord using a nasal endoscope. However, this setup creates spatial limitations as the nasal endoscope, light source equipment, and other treatment equipment are placed in a narrow space. Consequently, performing the procedure from various angles becomes challenging.

In addition, as the length of the optical fiber increases, the energy of the light on the optical fiber from the light source gradually decreases. As a result, the brightness of the light transmitted to the affected area may appears relatively weak. To address this issue, it becomes necessary to either turn off the surrounding lights or adjust the lighting brightness of the nasal endoscope accordingly.

Furthermore, the light-guided injection device presents additional challenges, including increased production costs associated with the main elements used, potential drug loss along the drug transmitting route, and difficulties related to equipment management and maintenance.
(Patent Document 0001) (Korean Patent Registration No. 10-1699229, Jan. 18, 2017)

### SUMMARY OF INVENTION

### Technical Problem

Therefore, in the related art, there exists a need for a light-guided injection with a novel design and configuration that can address the limitations and challenges associated with existing light-guided injection devices.

### Technical Solution

In order to address the aforementioned issues, an embodiment of the present invention introduces a portable light-guided injection device.

The portable light-guided injection device comprises several components, including a syringe part that injects drug; an injection needle part that invades an affected portion and injects the drug into the body; a main body part that connects the syringe part and the injection needle part to provide a drug transmitting path and has a light generating part configured to provide light to the affected portion; and an optical cable part that transmits the light provided from the light generating part to the affected portion.

In addition, the solution means of the above problems does not enumerate all the features of the present invention. Various features of the present invention, and its advantages and effects will be understood in more detail with reference to specific embodiments below.

### Advantageous Effects

The present invention addresses the limitations of existing light-guided injection devices and offers several advantageous effects. It provides a portable light-guided injection device that is user-friendly, easy to maintain, and cost-effective while minimizing light loss and drug loss during the procedure.

### BRIEF DESCRIPTION OF THE DRAWINGS

FIGS. 1 to 6 are views illustrating a portable light-guided injection device according to an embodiment of the present invention.

FIGS. 7 and 8 are views illustrating an injection needle part according to an embodiment of the present invention.

FIG. 9 is a view illustrating a main body part according to an embodiment of the present invention.

FIGS. 10 to 12 are views illustrating a 2-way connector part according to an embodiment of the present invention.

FIG. 13 is a view illustrating the 2-way connector part and a backflow prevention fixing part coupled thereto according to an embodiment of the present invention.

FIGS. 14 to 17 are views illustrating a light generating part according to an embodiment of the present invention.

FIG. 18 is a view illustrating a main body housing according to an embodiment of the present invention.

FIGS. 19 to 20 are views illustrating a syringe part according to an embodiment of the present invention.

FIG. 21 is a view illustrating some configurations of an optical cable part and a light generating part coupled thereto according to an embodiment of the present invention.

### BEST MODE FOR INVENTION

Hereinafter, preferred embodiments will be described in detail so that those skilled in the art may easily practice the present invention with reference to the accompanying drawings. However, in describing a preferred embodiments of the present invention in detail, if it is determined that a detailed description of a related known function or configuration may unnecessarily obscure the gist of the present invention, the detailed description will be omitted. In addition, the same reference numerals are used throughout the drawings for parts having similar functions and actions.

In addition, throughout the specification, when a part is said to be ‘connected’ to another part, this includes not only the case where it is ‘directly connected’, but also the case where it is ‘indirectly connected’ with another element therebetween. In addition, ‘including’ a certain component means that other components may be further included, rather than excluding other components unless otherwise stated.

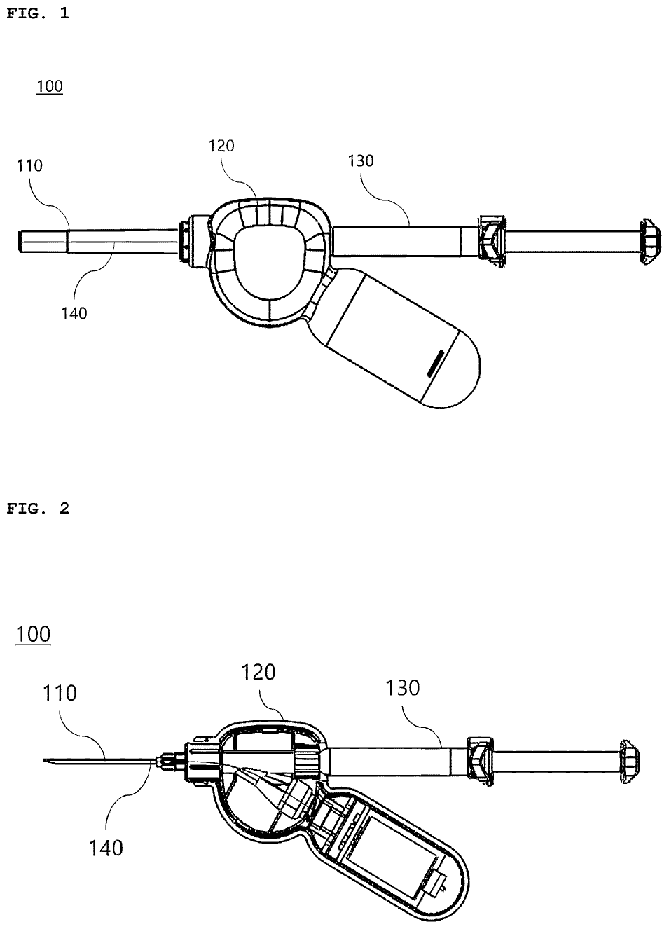

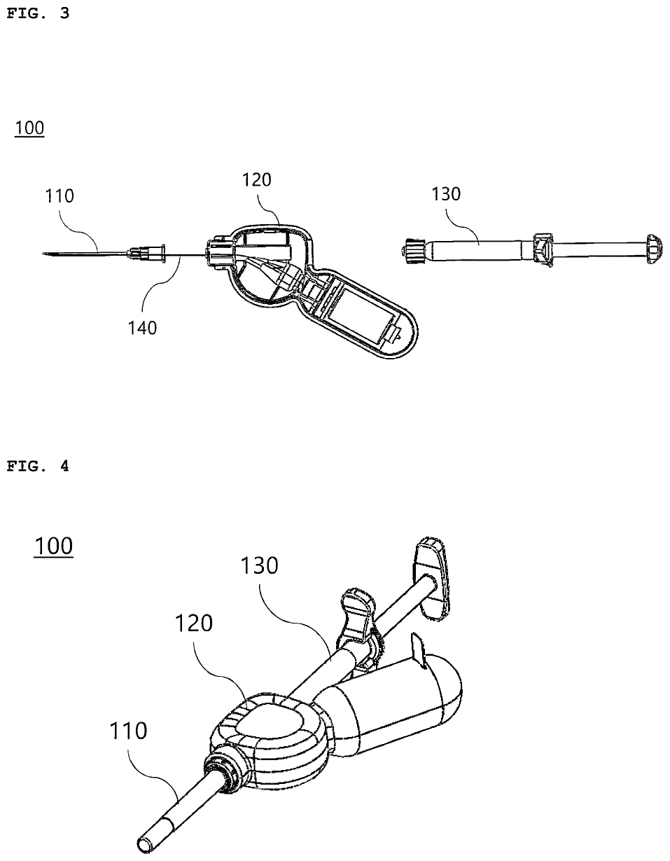

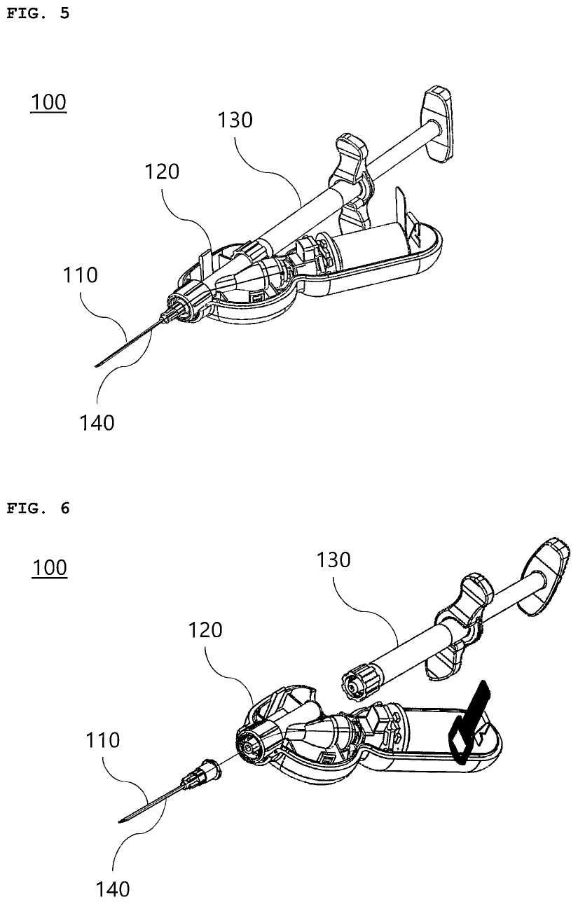

FIGS. 1 to 6 are views illustrating a portable light-guided injection device according to an embodiment of the present invention, and FIGS. 1 to 6 illustrates respectively a side view, a sectional view, an exploded sectional view, a stereoscopic view, a sectional stereoscopic view, and an exploded sectional stereoscopic view.

Referring to FIGS. 1 to 6, a portable light-guided injection device 100 according to an embodiment of the present invention may be configured to include an injection needle part 110, a main body part 120, a syringe part 130, and an optical cable part 140.

Here, the injection needle part 110 may invade the affected portion and inject the drug transmitted from the syringe part 130 into the body. Here, the drug to be injected may be, for example, filler, botox, steroid, silicone implant, etc., but is not necessarily limited thereto.

The main body part 120 connects the injection needle part 110 and the syringe part 130 to provide a drug transmitting path, and has a light generating part (not illustrated) configured to provide light to the affected portion, thus establishing a light transmitting path.

The syringe part 130 is for injecting the drug, and the optical cable part 140 transmits the light provided from the light generating part (not illustrated) to the affected portion.

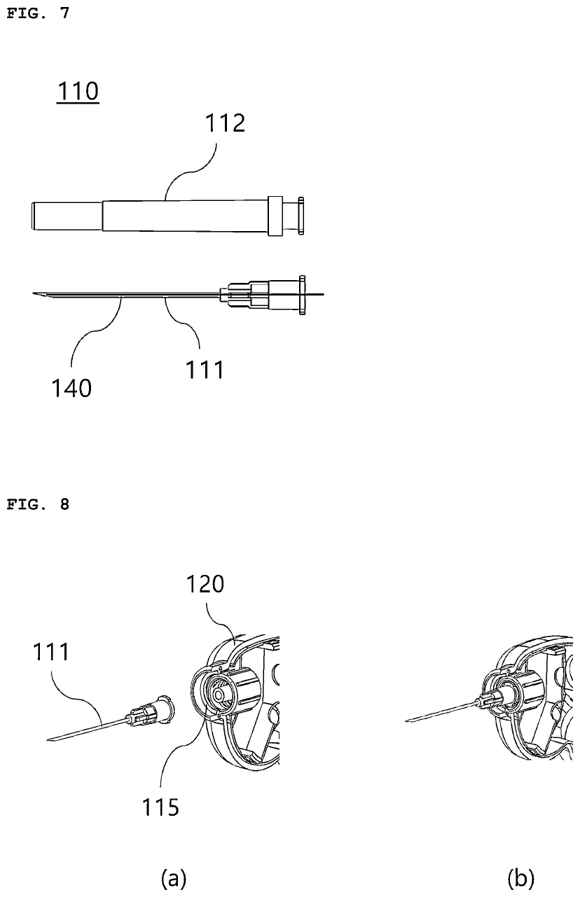

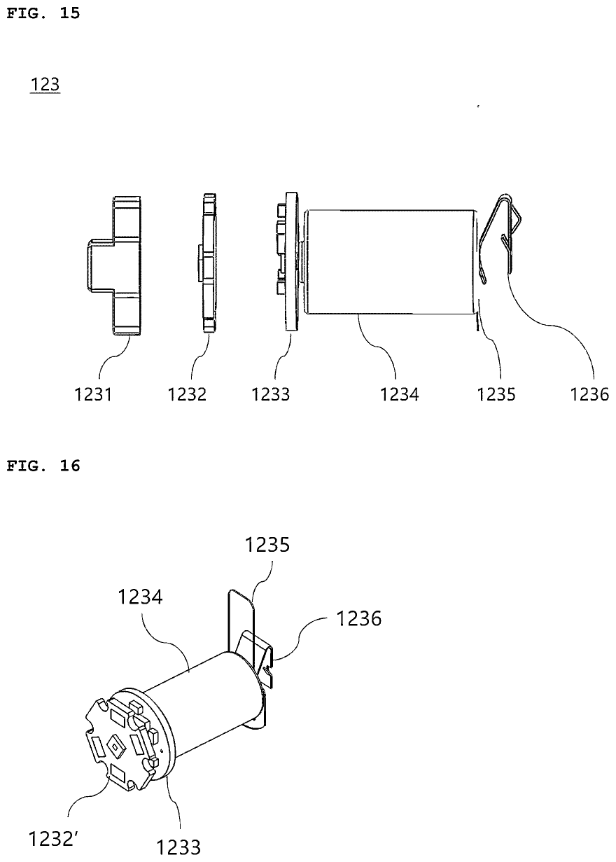

Hereinafter, each component of the portable light-guided injection device will be described in more detail with reference to FIGS. 7 to 21.

FIGS. 7 and 8 are views illustrating the injection needle part according to an embodiment of the present invention.

Referring to FIG. 7, the injection needle part 110 may include an injection needle 111 and an injection needle cap 112.

The injection needle 111 penetrates the affected portion and injects the drug into the body, and may have an outer diameter ranging from 0.305 mm to 1.270 mm and an inner diameter ranging from 0.140 mm to 0.838 mm. However, the size of the injection needle 111 is not necessarily limited thereto, and may be appropriately selected depending on the type of the drug to be injected into the body.

In addition, the optical cable part 140 to be described later is inserted through the hollow interior of the injection needle 111, through which light is transmitted to the affected portion.

The injection needle cap 112 is for protecting the injection needle 111 and preventing stabbing, and is coupled to the injection needle 111 to cover the outside of the injection needle 111, and may be removed from the injection needle 111 during the operating procedure.

Referring to FIG. 8, as illustrated in (a), the injection needle part 110 and the main body part 120 may be separated from each other, and the injection needle part 110 and the main body part 120 may be connected to each other by a luer lock 115 (see (b)).

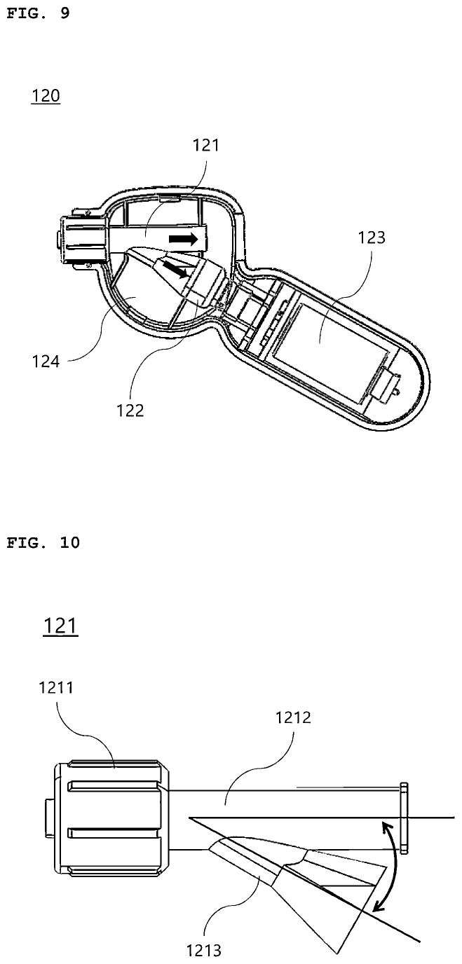

FIG. 9 is a view illustrating the main body part according to an embodiment of the present invention.

Referring to FIG. 9, the main body part 120 may include a 2-way connector part 121, a backflow prevention fixing part 122, a light generating part 123, and a main body housing 124.

Here, one end of the 2-way connector part 121 is connected to the injection needle part 110, a first direction (upper arrow in FIG. 9) of the other end is connected to the syringe part 130, and a second direction (lower arrow in FIG. 9) of the other end is connected to the light generating part 123 to provide both the drug transmitting path and the light transmitting path.

The backflow prevention fixing part 122 secures the optical cable part 140 and simultaneously prevents the drug injected from the first direction (drug injecting part) from flowing backward in the second direction (light source transmitting part).

The light generating part 123 generates the light to be provided to the affected portion.

The main body housing 124 forms an inner space that accommodates the 2-way connector part 121, the backflow prevention fixing part 122, and the light generating part 123.

FIGS. 10 to 12 are views illustrating the 2-way connector part according to an embodiment of the present invention.

Referring to FIGS. 10 to 12, the 2-way connector part 121 may include a first connector part 1211, a drug injecting part 1212, and a light source transmitting part 1213.

Here, the first connector part 1211 is connected to the injection needle part 110 and may be a luer lock type connector.

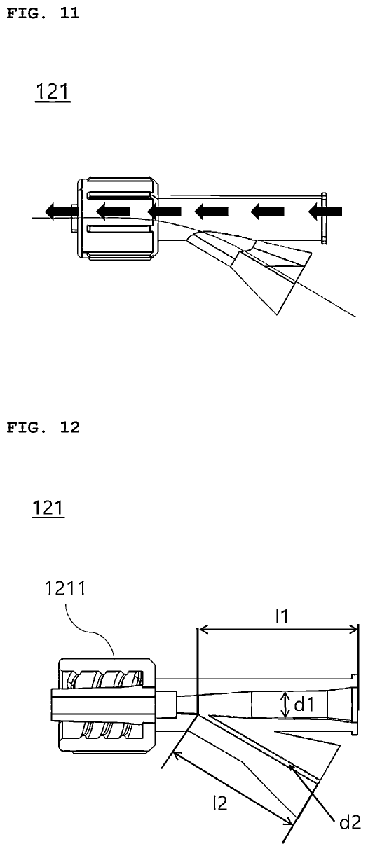

The drug injecting part 1212 accommodates the drug that is injected from the syringe part 130, providing the drug transmitting path (arrow in FIG. 11), and the light source transmitting part 1213 accommodates the optical cable part 140, which is inserted through the backflow prevention fixing part 122, providing the light transmitting path (solid line in FIG. 11).

In this case, the drug injecting part 1212 and the light source transmitting part 1213 may be designed to form an acute angle (i.e., an angle smaller than) 90° with each other, preferably the angle formed by the drug injecting part 1212 and the light source transmitting part 1213 may ranges from 20° to 80°. By doing so, it is possible to facilitate insertion of the optical cable part 140 and to prevent bending of the optical cable part 140.

In addition, to minimize drug loss, the length 11 of the drug injecting part 1212 may be designed to be shorter than the length 12 of the light source transmitting part 1213.

Moreover, by designing the inner diameter d2 of the light source transmitting part 1213 smaller than the inner diameter d1 of the drug injecting part 1212, it is possible to prevent drug backflow and minimize drug loss during drug injection. According to an embodiment, the inner diameter d2 of the light source transmitting part 1213 can be matched to the outer diameter of the optical cable part 140 that is inserted into the light source transmitting part 1213.

The aforementioned 2-way connector part 121 may be made of an opaque material to prevent light scattering.

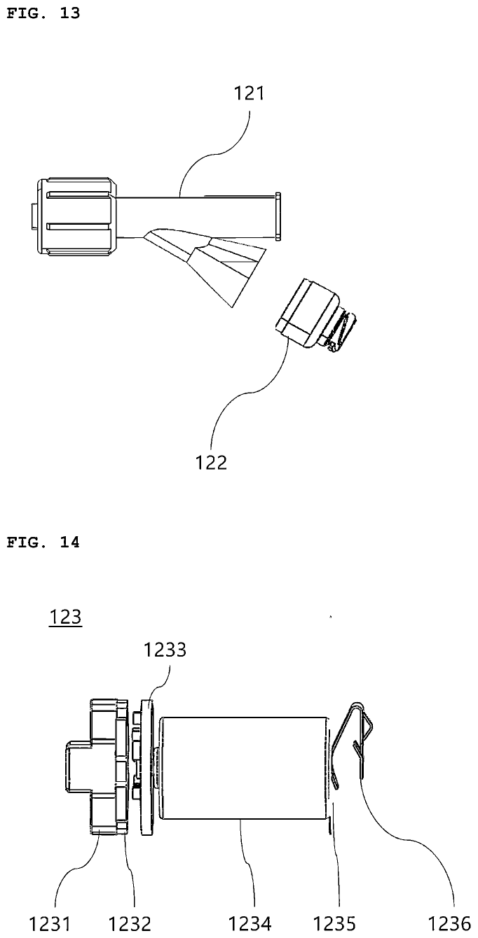

FIG. 13 is a view illustrating the 2-way connector part and the backflow prevention fixing part coupled thereto according to an embodiment of the present invention.

Referring to FIG. 13, the backflow prevention fixing part 122 is coupled to the end of the light source transmitting part 1213 of the 2-way connector part 121 to secure the optical cable part 140 inserted into the light source transmitting part 1213 and prevent the drug backflow.

The backflow prevention fixing part 122 may be configured, for example, in various forms such as such as a STEM swabable silicone valve, a silicone sealing, a fixing cap, and a directly fixing method using an adhesive like epoxy.

In addition, the backflow prevention fixing part 122 may be integrated with a light source cap part (1231 in FIG. 14) to be described later.

FIGS. 14 to 17 are views illustrating the light generating part according to an embodiment of the present invention.

Referring to FIGS. 14 to 17, the light generating part 123 may include the light source cap part 1231, a light source 1232, a heat sink 1232′, a control board 1233, a power supply part 1234, a switch part 1235, and a contact plate 1236.

The light source cap part 1231 may be coupled to the light source 1232 to prevent scattering of the light, and may ensure proper alignment and fixation of the centers of the light source 1232 and the optical cable part 140. The light source cap part 1231 may be, for example, a connector such as SMA, FC, and ST, or a ceramic connector, but is not necessarily limited thereto.

The light source 1232 generates light when power is applied. The light source 1232 may be, for example, a laser diode, or LED, but is not necessarily limited thereto.

In addition, the control board 1233 and the heat sink 1232′ may be selectively provided according to the type of the light source 1232 used. Here, the control board 1233 is provided for adjusting the applied voltage, while the heat sink 1232′ is provided for dissipating the heat generated by the light source 1232, and the control board 1233 and the heat sink 1232′ may be implemented, for example, using a PCB board.

The power supply part 1234 supplies power to the light source 1232.

The switch part 1235 is provided for controlling power supply from the power supply part 1234.

According to an embodiment, as illustrated in FIG. 16, the switch part 1235 is designed as a thin plate located between the power supply part 1234 and the contact plate 1236 to establish or break the connection between the power supply part 1234 and the contact plate 1236.

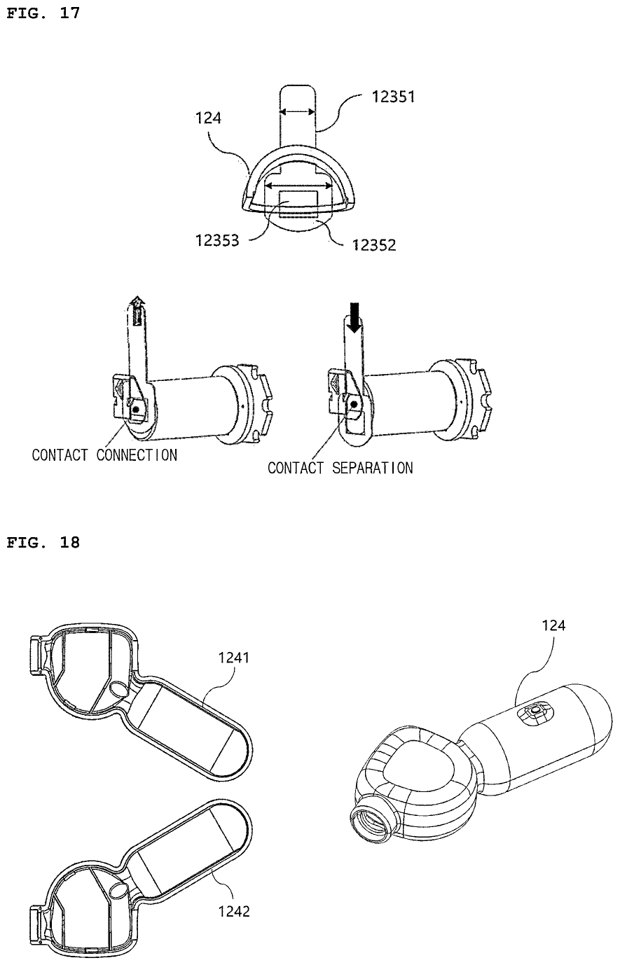

Specifically, as illustrated in FIG. 17, the switch part 1235 may include a handle part 12351, a head part 12352, and a connection hole 12353 situated within the head part 12352.

The handle part 12351 is a portion that protrudes from the main body housing 124 and may be gripped to push and pull the switch part 1235.

The head part 12352 is a portion that extends from the handle part 12351 to be integrally formed, and is inserted into the main body housing 124 to be positioned between the power supply part 1234 and the contact plate 1236.

The connection hole 12353 is formed in a specific area of the head part 12352, and as the switch part 1235 moves, the connection hole 12353 enables the power supply part 1234 and the contact plate 1236 to establish a connection.

Accordingly, the power supply part 1234 supply power to the light source 1232, resulting in the generation of light.

A switch hole (not illustrated) is incorporated into the main body housing 124 so that the handle part 12351 integrally formed with the head part 12352 extends outside of the main body housing 124 through the switch hole (not illustrated).

The width of the handle part 12351 may be designed to be narrower than the width of the head part 12352, the width of the handle part 12351 may be narrower than the width of the switch hole (not illustrated), and the width of the head part 12352 may be made wider than the width of the switch hole (not illustrated). In this case, since the switch part 1235 is not separated from the main body housing 124 to the outside, it is possible to prevent loss thereof.

The configuration of the switch part 1235 described above with reference to FIG. 17 is only an example, and the switch part 1235 may be modified in various forms.

FIG. 18 is a view illustrating a main body housing according to an embodiment of the present invention.

Referring to FIG. 18, the main body housing 124 is composed of a first housing 1241 covering one surface of the main body part and a second housing 1242 covering the other surface of the main body part, and the first housing 1241 and the second housing 1242 are coupled together to form a space therein to accommodate the 2-way connector part 121, the backflow prevention fixing part 122, and the light generating part 123.

In addition, as described above with reference to FIG. 17, the switch hole (not illustrated) may be formed in a portion corresponding to the area where the power supply part 1234 and the contact plate 1236 are connected to each other within the main body housing 124.

Furthermore, the main body housing 124 is designed with an ergonomic shape to ensure comfortable and convenient handling for users when the device is used in its portable form.

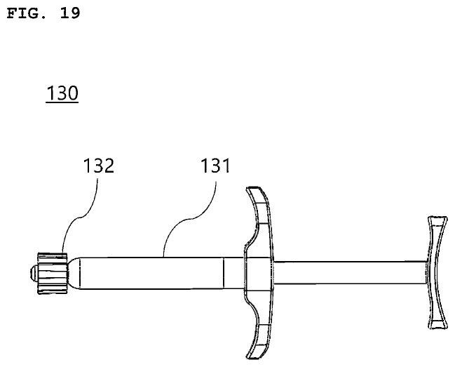

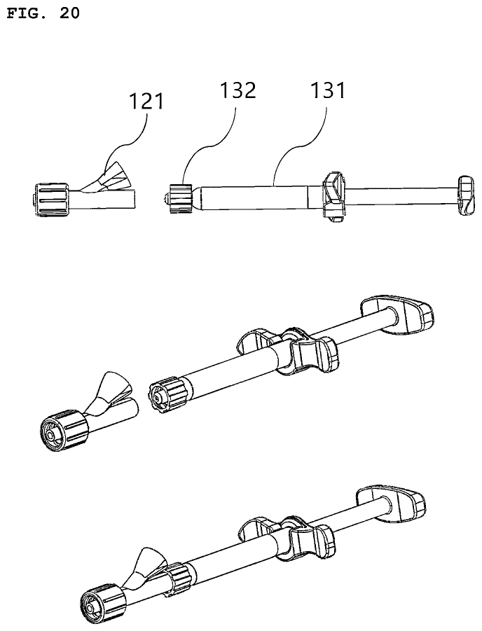

FIGS. 19 and 20 are views illustrating the syringe part according to an embodiment of the present invention.

Referring to FIGS. 19 and 20, the syringe part 130 may include a syringe 131 and a second connector part 132.

Here, the second connector part 132 connects the syringe 131 and the 2-way connector part 121, and may be a luer lock type connector.

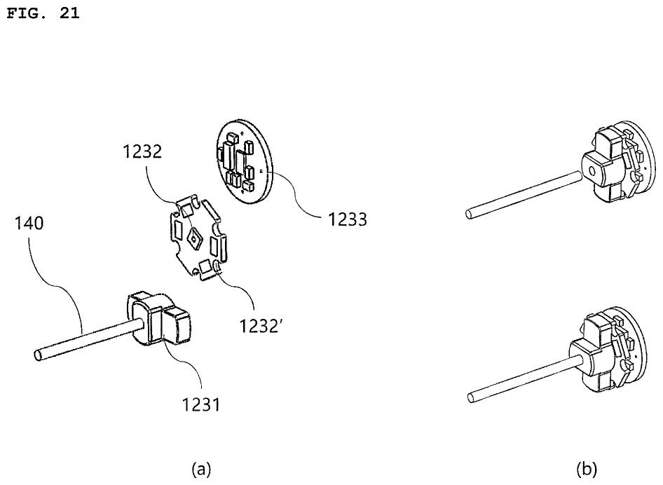

FIG. 21 is a view illustrating some configurations of the optical cable part and the light generating part coupled thereto according to an embodiment of the present invention.

Referring to FIG. 21, the optical cable part 140 serves as a medium for transmitting light generated from the light source 1232, and may be implemented using various materials, for example, PMMA (plastic-based), silica (glass-based) optical fibers, or the like.

The optical cable part 140 may be connected to or detached from the light source cap part 1231. For example, the optical cable part 140 may be fixed to the light source cap part 1231 using adhesive or may be connected thereto connectors such as SMA or FC.

The diameter of the optical cable part 140 may range from 10 μm to 200 μm, but is not necessarily limited thereto.

In addition, the optical cable part 140 may be inserted into the injection needle 111 through the backflow prevention fixing part 122, the light source transmitting part 1213, and the first connector part 1211 to transmit light to the affected portion.

The present invention is not limited by the foregoing embodiments and accompanying drawings. It will be clear to those skilled in the art that the components according to the present invention may be substituted, modified, and changed without departing from the technical spirit of the present invention.

1. A portable light-guided injection device comprising:
a syringe part that injects drug;
an injection needle part that invades an affected portion and injects the drug into the body;
a main body part that connects the syringe part and the injection needle part to provide a drug transmitting path and has a light generating part configured to provide light to the affected portion; and
an optical cable part that transmits the light provided from the light generating part to the affected portion.

2. The portable light-guided injection device of claim 1, wherein the main body part includes
a 2-way connector part of which one end is connected to the injection needle part, a first direction of the other end is connected to to the syringe part, and a second direction of the other end is connected to the light generating part to provide a drug transmitting path and a light transmitting path;
a backflow prevention fixing part that is coupled to an end of the 2-way connector part in the second direction to fix the optical cable part and prevent a backflow of the drug; and
a main body housing that accommodates the light generating part, the 2-way connector part, and the backflow prevention fixing part in an inner space.

3. The portable light-guided injection device of claim 2, wherein the 2-way connector part includes
a first connector part that is connected to the injection needle part;
a drug injecting part that accommodates the drug injected from the syringe part and provides a drug transmitting path; and
a light source transmitting part that accommodates the optical cable part inserted through the backflow prevention fixing part and provides a light transmitting part.

4. The portable light-guided injection device of claim 3, wherein the drug injecting pat and the light source transmitting part are formed to form an acute angle with each other.

5. The portable light-guided injection device of claim 3, wherein a length of the drug injecting part is formed shorter than a length of the light source transmitting part.

6. The portable light-guided injection device of claim 3, wherein an inner diameter of the light source transmitting part is formed smaller than an inner diameter of the drug injecting part.

7. The portable light-guided injection device of claim 3, wherein the 2-way connector part is formed of a material of opaque color.

8. The portable light-guided injection device of claim 2, wherein the light generating part includes
a light source that generates light;
a power supply part that applies power to the light source;
a switch part that controls power supply by the power supply part; and
a light source cap part that is coupled to the light source to prevent scattering of the light source and aligns and fixes centers of the light source and the optical cable part.

9. The portable light-guided injection device of claim 8, wherein the light generating part further includes at least one of
a control board that adjusts a voltage applied to the light source; and
heat sinks that dissipate heat generated in the light source.

10. The portable light-guided injection device of claim 8, wherein the switch part is formed in a plate shape located between the power supply part and the contact plate to connect or disconnect between the power supply part and the contact plate.

11. The portable light-guided injection device of claim 10, wherein the switch part includes
a handle part that protrudes to the outside of the main body housing and is configured to hold the switch portion to push and pull the switch portion;
a head part that extends from the handle part and is integrally formed, and inserted into the main body housing to be positioned between the power source part and the contact plate; and
a connection hole that is formed in a partial region of the head part, and
the power supply part and the contact plate are connected to each other through the connection hole as the switch part moves.

12. The portable light-guided injection device of claim 11, wherein a switch hole is formed in the main body housing so that the handle part protrudes to the outside of the main body housing through the switch hole, and
a width of the handle part is formed smaller than a width of the head part, the width of the handle part is formed smaller than a width of the switch hole, and the width of the head part is formed larger than the width of the switch hole.

13. The portable light-guided injection device of claim 2, wherein the syringe part includes
a syringe; and
a second connector part that connects the syringe and the 2-way connector part.

14. The portable light-guided injection device of claim 8, wherein the optical cable part is coupled to or separated from the light source cap part, and
the optical cable part is inserted into the injection needle through the backflow prevention fixing part, the light source transmitting part, and the first connector part to transmit light to the affected portion.
